(19) United States
(12) Patent Application Publication
(10) Pub. No.: US 2023/0248491 A1
(43) Pub. Date: Aug. 10, 2023

(54) COMPOSITIONS AND METHODS FOR ADDITIVE MANUFACTURING OF ZrO2-BASED CERAMIC DENTAL CROWNS

(71) Applicant: NUtech Ventures, Lincoln, NE (US)

(72) Inventors: Bai Cui, Lincoln, NE (US); Nathan Snyder, Lincoln, NE (US); Kevin Zhao, Lincoln, NE (US); Lanh Trinh, Lincoln, NE (US)

(21) Appl. No.: 18/166,229

(22) Filed: Feb. 8, 2023

Related U.S. Application Data
(60) Provisional application No. 63/307,959, filed on Feb. 8, 2022.

(51) Int. Cl. A61C 13/00; A61C 5/77; A61C 13/083; A61C 13/15

(52) U.S. Cl. CPC A61C 13/0019; A61C 5/77; A61C 13/0835; A61C 13/0018; A61C 19/003

(57) ABSTRACT

Systems and methods for additive manufacturing of ZrO₂ ceramic objects, and in particular ZrO₂-based ceramic dental crowns. A method includes mixing a doped ZrO₂ powder with a photo-curable resin to produce a printing mixture, 3D printing of a dental crown green body structure using the printing mixture, wherein the 3D printing includes an advanced digital light processing (ADLP) process using a gradient printing technique to form a color gradient in the dental crown green body structure, debinding the dental crown green body structure to remove organic polymers using one of a thermal debinding process, an infrared debinding process, and a laser debinding process, sintering the debound dental crown structure to produce a densified dental crown structure using one of a pressureless sintering process, a laser sintering process, and an electric field sintering process, and surface engineering the densified dental crown structure to produce a finished dental crown structure using one of a mechanical polishing process, a laser polishing process, a laser shock peening process, a shot peening process, and a water jet process.

### CROSS REFERENCE TO RELATED APPLICATION

This patent application claims priority to U.S. Provisional Patent Application No. 63/307,959, entitled “COMPOSITIONS AND METHODS FOR ADDITIVE MANUFACTURING OF ZrO₂-BASED CERAMIC DENTAL CROWNS,” filed Feb. 8, 2022, which is incorporated herein by reference in its entirety.

### BACKGROUND

The present disclosure provides compositions, systems and methods for additive manufacturing of ZrO₂-based ceramic dental crowns.

Advanced treatment methods of dental crowns and bridges using ZrO₂-based ceramics are starting to replace traditional methods using metals or porcelains fused to metals. Ceramic materials are predicted to occupy the larger share in the market compared to metals due to many advantages, such as matching the color of natural teeth. ZrO₂-based ceramics are the most promising ceramic materials for dental crowns due to their high strength and hardness, corrosion resistance, color match with the human tooth, and excellent compatibility with the human body environment.

ZrO₂ is an excellent biocompatible ceramic material, which has high mechanical performance and good wear resistance, and has no cytotoxic effect when tested both in vitro and in vivo for a long time.

The traditional (conventional) method for fabrication of ZrO₂ dental crowns is a subtractive manufacturing process, which uses a computer-aided design and computer-aided manufacturing (CAD-CAM) or a computer numerical control (CNC) machining system. In this process, a designer in the dental clinic first studies the color and transparency requirements of the patient and identify the type and amount of ZrO₂ powders to meet the requirements. Then, a crown model is designed using the CAD software, and the CAM/CNC machining parameters are determined based on the 3D dental model. A ZrO₂ block is sintered using ZrO₂ powders at high temperatures (˜1500° C.) in a furnace, and is finally machined to the dental crowns using the CAM/CNC machining. The CAD-CAM technology has advantages in terms of the application of new materials, reduced labor, cost effectiveness, and quality control. However, the problems of CAD-CAM technology include: 1) a complex model is difficult to form using this method; 2) a considerable amount of ZrO₂ material is wasted; 3) a high designer cost represented about 60% of the overall cost; and 4) a lengthy processing time causing a very long turnaround time of 7 days on average.

There is a critical need for improved methods of manufacturing ZrO₂-based ceramic dental crowns.

### SUMMARY

The present embodiments provide compositions, systems and methods for additive manufacturing of ZrO₂ dental crowns, and in particular ZrO₂-based ceramic dental crowns.

According to an embodiment, a method of manufacturing a ZrO₂-based ceramic dental crown is provided. The method includes mixing a doped ZrO₂ powder with a solvent and a dispersant to produce a slurry, drying the slurry to remove the solvent and produce a dried ceramic powder, mixing the dried ceramic powder with a photo-curable resin to produce a printing mixture, and 3D printing a dental crown green body structure using the printing mixture, wherein the 3D printing includes an advanced digital light processing (ADLP) process. The doped ZrO₂ powder is selected from the group consisting of Powder A, Powder B, Powder C and Powder D, wherein Powder A contains 2 to 4 mol. % yttrium (Y)-doped zirconia, wherein Powder B contains 4 to 8 mol. % yttrium (Y)-doped zirconia, wherein Powder C contains zirconia doped with 2 to 4 mol. % yttrium (Y) and 0.1 to 1.0 mol. % iron (Fe), and wherein Powder D contains zirconia doped with one or more of magnesium (Mg), calcium (Ca), cerium (Ce), aluminum (Al), titanium (Ti), germanium (Ge), scandium (Sc), or tin (Sn).

In certain aspects, the photo-curable resin is selected from the group consisting of Genesis flexible development base resin (Tethon 3D), 1,6-hexanediol diacrylate (HDDA), polyethylene glycol diacrylate (PEGDA), and diphenyl (2,4,6-trimethylbenzoyl) phosphine oxide (TPO).

In certain aspects, the dispersant is selected from the group consisting of 1-10 wt. % oleic acid (OA), DISPERBYK-103, DISPERBYK-111, DISPERBYK-180, DISPERBYK-2008, DISPERBYK-2013, DISPERBYK-2155, TEGO Dispers 685, and polyvinyl pyrrolidone K15 (PVP-K15).

According to certain aspects, the 3D printing includes a gradient printing process.

According to an embodiment, the method further includes debinding the dental crown green body structure to remove organic polymers, including the resin and the dispersant, and to produce a debound dental crown structure using one of a thermal debinding process, an infrared debinding process, and a laser debinding process.

According to an embodiment, the method further includes sintering the debound dental crown structure to produce a densified dental crown structure using one of a pressureless sintering process, a laser sintering process, and an electric field sintering process.

According to an embodiment, the method further includes surface engineering the densified dental crown structure to produce a finished dental crown structure using one of a mechanical polishing process, a laser polishing process, a laser shock peening process, a shot peening process, and a water jet process.

According to an embodiment, the ADLP process includes printing the dental crown green body structure using two or more different printing mixtures, each different printing mixture including one of Powder A, Powder B, Powder C or Powder D, using a gradient printing technique to form a color gradient in the dental crown green body structure.

According to an embodiment, an ADLP system is provided for manufacturing an object. The ADLP system includes a resin tank structure including at least two separate resin vats, each of the at least two separate resin vats holding a different printing mixture comprising a photo-curable resin and a different powder, a build platform configured to hold a green body object during a printing operation, movement components configured to controllably move the build platform relative to the resin tank structure in vertical and horizontal directions, and a light source configured to project a light pattern through a bottom portion of the resin vat structure. During the printing operation, a first layer of the green body object is cured in a first one of the at least two separate resin holding vats, and a second layer of the green body object is cured in a second one of the at least two separate resin holding vats. In certain aspects, the different powders may include ZrO₂ powders as described herein. In certain aspects, the light may include UV light or light of another wavelength range.

According to an embodiment, an ADLP system for manufacturing a ZrO₂-based ceramic object is provided. The ADLP system includes a resin tank structure including at least two separate resin vats, each of the at least two separate resin vats holding a different printing mixture comprising a photo-curable resin and a different doped ZrO₂ powder, a build platform configured to hold a green body object during a printing operation, movement components configured to controllably move the build platform relative to the resin tank structure in vertical and horizontal directions, and a UV light source configured to project a UV light pattern through a bottom portion of the resin vat structure. During the printing operation, a first layer of the green body object is cured in a first one of the at least two separate resin holding vats, e.g., according to a first UV light pattern projected by the UV light source, and a second layer of the green body object is cured in a second one of the at least two separate resin holding vats, e.g., according to a second UV light pattern projected by the UV light source.

In certain aspects, the movement components include an elevator structure configured to move the build platform in the vertical direction, and a resin tank guide structure configured to move the resin tank structure in a horizontal direction.

In certain aspects, the ADLP system further includes a controller configured to control the movement components and the UV light source.

In certain aspects, the resin tank structure includes at least three separate resin vats.

In certain aspects, each different doped ZrO₂ powder includes a doped ZrO₂ powder selected from the group consisting of Powder A, Powder B, Powder C and Powder D, wherein Powder A contains 2 to 4 mol. % yttrium (Y)-doped zirconia, wherein Powder B contains 4 to 8 mol. % yttrium (Y)-doped zirconia, wherein Powder C contains zirconia doped with 2 to 4 mol. % yttrium (Y) and 0.1 to 1.0 mol. % iron (Fe), and wherein Powder D contains zirconia doped with one or more of magnesium (Mg), calcium (Ca), cerium (Ce), aluminum (Al), titanium (Ti), germanium (Ge), scandium (Sc), and or tin (Sn).

In certain aspects, the photo-curable resin is selected from the group consisting of Genesis flexible development base resin (Tethon 3D), 1,6-hexanediol diacrylate (HDDA), polyethylene glycol diacrylate (PEGDA), and diphenyl (2,4,6-trimethylbenzoyl) phosphine oxide (TPO).

In certain aspects, the UV light source configured to project a UV light pattern includes a source of UV radiation or light, a projector screen, which projector may include a digital micrometer device including tens or hundreds or thousands or millions of micro mirrors that controllably navigate the light to create the UV light pattern projected onto the bottom of the resin tank structure.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings. In the drawings, like reference numbers indicate identical or functionally similar elements.

### BRIEF DESCRIPTION OF THE DRAWINGS

FIG. 1 illustrates a process for manufacturing ZrO₂-based ceramic dental crowns, according to an embodiment.

FIG. 2 is a schematic diagram of an Advanced Digital Light Processing (ADLP) printing process setup according to an embodiment.

FIG. 3 is a schematic diagram of a resin vat array including three vats according to an embodiment.

FIG. 4 illustrates a 3D model of a human dental crown in panel (A), a 3D printed dental crown processed with the ADLP process using a slurry containing Powder A in panel (B), and a sintered dental crown after debinding and sintering processes in panel (C), according to embodiments.

### DETAILED DESCRIPTION

The present disclosure provides compositions, systems and methods for additive manufacturing of ZrO2 dental crowns, and in particular ZrO2-based ceramic dental crowns.

In certain aspects, the present embodiments provide an additive manufacturing process that reduces or minimizes the designer cost, decreases material waste, and significantly reduces the processing and turnaround time for manufacturing ZrO₂-based ceramic dental crowns.

Doping is an effective approach to optimize the crystal structures as well mechanical and optical properties of ZrO₂. For example, 3 mol. % yttrium-doped tetragonal zirconia polycrystal (Y-TZP) exhibits a high flexural strength (900-1200 MPa) and fracture toughness (9-10 MPa m^{1/2}) due to the unique transformation toughening mechanisms. High-strength Y-TZP has been used in dental tissue replacement such as root canal posts, frameworks for all-ceramic crowns and fixed dental prostheses.

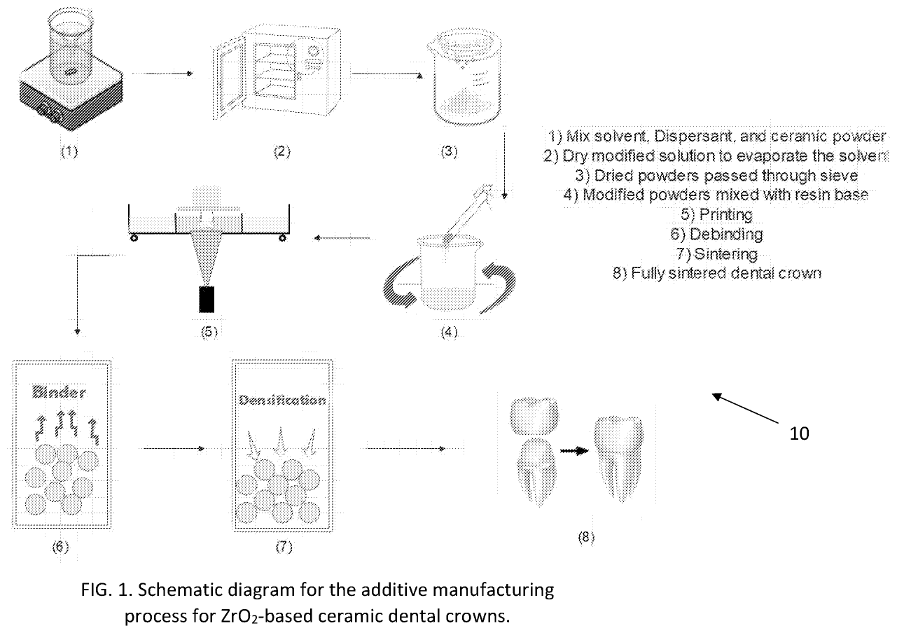

FIG. 1 illustrates a process 10 for manufacturing ZrO₂-based ceramic dental crowns, according to an embodiment, including steps of slurry mixing, advanced digital light processing (ADLP), de-binding, sintering, and surface engineering. In step 1, the solvent, dispersant and ceramic powder are mixed together, and in step 2 the solution is dried to remove the solvent. In step 3, the dried powder mixture is passed through a sieve and in step 4, the powders are mixed with a resin base. In step 5, the object, e.g., dental crown, is printed, in step 6, the object is subjected to a debinding process to remove dispersant and resin material, and in step 7, the debound object is sintered to densify the object. Step 8 shows the fully sintered dental crown being placed over a (reshaped) tooth.

### Chemical Compositions

According to certain embodiments, the chemical compositions of ceramic dental crowns include ZrO₂ doped with organic and inorganic additives. A starting ceramic powder includes doped zirconia (ZrO₂) and may include one of the following compositions, but is not limited to:

A. Powder A contains 2 to 4 mol. % yttrium (Y)-doped zirconia, which stabilizes the tetragonal crystal structure. The dental crowns additively manufactured with Powder A have a high flexural strength, a high fracture toughness, and an opaque and white color.

B. Powder B contains 4 to 8 mol. % yttrium (Y)-doped zirconia, which has a cubic crystal structure. The dental crowns additively manufactured with Powder B have a translucent and white color, and lower flexural strength and fracture toughness than those manufactured with Powder A.

C. Powder C contains zirconia doped with 2 to 4 mol. % yttrium (Y) and 0.1 to 1.0 mol. % iron (Fe), which has a tetragonal crystal structure. The dental crowns additively manufactured with Powder C have an opaque and light yellow color, and similar flexural strength and fracture toughness to those manufactured with Powder A.

D. Powders with other zirconia dopants including but not being limited to one or more of: magnesium (Mg), calcium (Ca), cerium (Ce), aluminum (Al), titanium (Ti), germanium (Ge), scandium (Sc), and tin (Sn).

In an embodiment, as shown in FIG. 1, a first step of the fabrication process includes slurry mixing. In an embodiment, the slurry includes a mixture of doped ZrO₂ powders, an ultraviolet (UV)-curable resin containing UV-initiators, and a dispersant. The doped ZrO₂ powders may be any one powder of powders A to D, above. The photo-curable resin includes but is not limited to: Genesis flexible development base resin (Tethon 3D), 1,6-hexanediol diacrylate (HDDA), polyethylene glycol diacrylate (PEGDA), or diphenyl (2,4,6-trimethylbenzoyl) phosphine oxide (TPO). The dispersant includes but is not limited to 1-10 wt. % oleic acid (OA), DISPERBYK-103, DISPERBYK-111, DISPERBYK-180, DISPERBYK-2008, DISPERBYK-2013, DISPERBYK-2155, TEGO Dispers 685, or polyvinyl pyrrolidone K15 (PVP-K15).

The slurry is mixed by a mixing method that includes but is not limited to: magnetic stir mixing, shear mixing, and ball milling. The mixing speed may be between 100 to 2000 rpm, for example. The temperature of the slurry during mixing should be from about −30° C. to about 80° C.

### ADLP/Gradient Printing

According to certain embodiments, an ADLP procedure including a gradient printing technique is provided, which enables ZrO₂ dental crowns with gradient color changes similar to the human tooth.

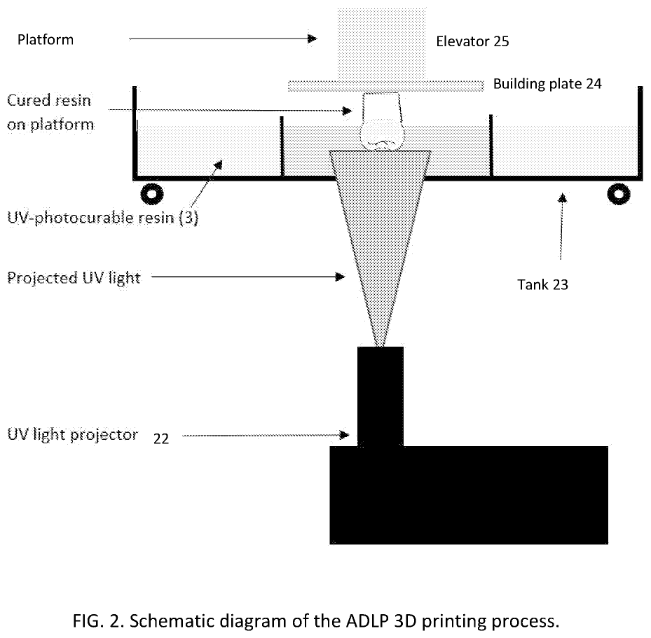

Digital light processing (DLP) is a 3D printing process traditionally used for polymer materials and recently applied to ceramic materials. Compared to other 3D printing techniques for ceramic materials, such as stereolithography or binder jetting, DLP has the advantages of fast printing speed, relatively high resolution, and allows for using resins with a high solid load of ceramic particles (e.g., 40˜80%). The DLP method originated from image projection technology, which uses a UV light of a specific wavelength and a UV-curable liquid resin to produce 3D-printed solid products (see, e.g., FIG. 2). As shown in FIG. 2, the main components of DLP 3D printing include a digital projector screen 22 including micro mirrors (to control the directivity/locations of the curing light), a resin tank guide to control the projected micro mirror illumination pattern based on the 3D model, a resin tank 23, a build plate 24, and an elevator 25 attached to build plate 24 for vertical movement of the build plate 24, and a controller (not shown) including a processor and associated memory to control the various components including the UV source. During the 3D printing process, a 3D model (e.g., Standard Tessellation Language format (STL) file) is uploaded to the printer, and the UV-curable resin containing ceramic powders is filled into the resin tank. The 3D printing begins by curing a layer of resin in a controlled manner with the UV light to form a solid layer attached to the build platform, and then the build platform moves up a distance of one-layer thickness to cure another layer. For each layer, the curing light is guided or steered in a path representing that layer of the component being printed. For example the digital projector screen, in conjunction with the micro mirrors in a digital micrometer device, flashes an image or pattern of the layer at the same time. Such process is repeated layer by layer until the whole part is complete to form a “green body” that contains the ceramic powders in the cured polymer resin.

The present embodiments provide an ADLP process to further modify the traditional DLP process system to satisfy the patients' need to print the dental crowns with gradient color changes similar to the human tooth. In the present ADLP process, the STL file of the dental crown of a patient is first imported into the ADLP system. Compared to the traditional DLP process that uses only a single resin vat, in an embodiment, the ADLP process uses a resin vat array including more than one vat, e.g., two or three or four or more vats.

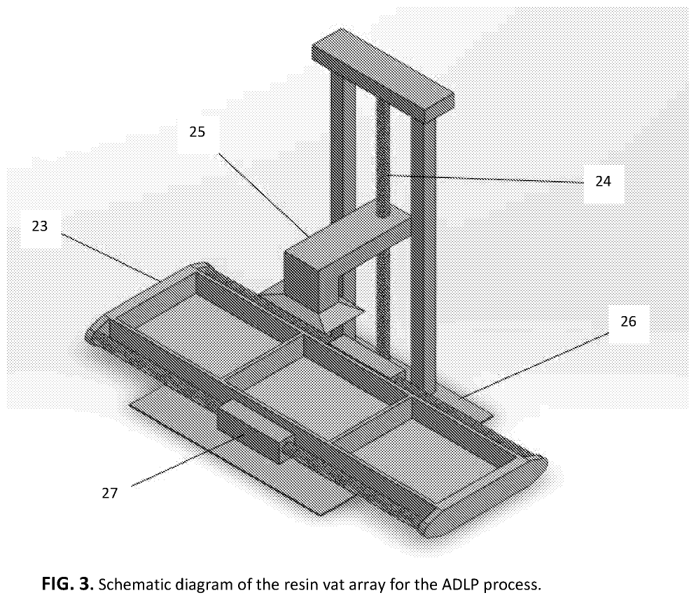

FIG. 3 is a schematic diagram of a resin tank 23 with a resin vat array including three vats according to an embodiment. The resin vat array holds the different types of slurries in each vat (e.g., containing Powders A, B, and C, respectively) for the 3D printer to change between or select between, which is accomplished by laterally shifting the resin tank 23 between cured layers until the desired resin vat is positioned under the Build Platform 25. The Build Platform 25 is the component that the green body is adhered to, which is lowered into the resin vats where the UV projector cures the subsequent layers before raising up and repeating the process until a full green body is printed. The Platform Elevator 24 controls and guides the up-and-down motion of the Build Platform 25. The Chamber Base 26 supports the components within the printing chamber. The resin tank guide 27 guides the lateral movement of the resin tank 23. The material types for the resin tank and vat array include but are not limited to: stainless steels (e.g., 304, 309, 316, 321, 410, 420, 430, 17-4 PH), nickel alloys (e.g., 22, 600, 625, 718, C-276), and aluminum alloys (e.g., 2024, 3003, 6061, 6063, 5086, 5083). In an embodiment, the dimensions of the each resin vat tray are from 5×5×1 to 50×50×10 cm. Linear actuators and drive motors may be used to adjust the relative movement of the vats and build platform, and for the elevator motion and the resin tank guide motion.

In an embodiment, to print the dental crowns with gradient color changes, Resin Vat Array 1 holds the different types of slurries e.g., (containing Powders A, B, and C, respectively) in each vat. For example, the layers with an opaque and white color are printed using the resin vat containing Powder A, the layers with a translucent and white color are printed using the resin vat containing Powder B, and the layers with an opaque and light yellow color are printed using the resin vat containing Powder C. The Build Platform 2 is moved between each vat to allow the printing of layers with gradient color changes.

For example, in one scenario, if the main body of a patient's dental crown has the opaque and white color while the edge is more translucent, the Build Platform 2 will be on the resin vat containing Powder A to print the main body and then move to the resin vat containing Powder B to print the edge. In another scenario, if a patient's dental crown has the color change from the light yellow on the bottom to more white on the top, the Build Platform 2 will be on the resin vat containing Powder C to print the bottom and then move to the resin vat containing Powder A to print the top of dental crown. In the case that the surface of the green body is not clean, one of the vats in the array can also contain cleaning fluids (including but is not limited to: ethanol, isopropyl alcohol) to clean the surface of the green body between the cured layers.

Many ADLP components and parameters are important for the quality control of ZrO₂-based ceramic dental crowns, enabling the formation of the optimized microstructures and outstanding mechanical and optical properties. According to certain embodiments, ADLP components and parameters of note may include:

(1) The light source for curing the resin may include a broadband or narrowband UV light source, a UV laser, xenon lamps, and a light-emitting diode, with a wavelength of from about 10 nm to about 420 nm and a radiant light power from about 1 W to about 100 W.

(2) The layer thickness is from about 1 μm to about 100 μm.

(3) The printing direction (the angle between the layer normal and the light beam) is from 0° to 90°, depending on the orientation of the model during the initial slicing.

(4) Exposure intensity is from about 1 to about 50 mW cm′.

(5) Basic/Initial exposure time is from about 0.01 to about 600 seconds.

(6) Temperature is from about −30 to about 80° C.

(7) The dimensions of the as-printed green body of dental crowns are from roughly 1×1×1 mm³ to about 50×50×50 mm³.

(8) The printing time for each dental crown is from about 1 minute to about 24 hours.

### Infrared Debinding and Laser Debinding

According to certain embodiments, two novel debinding methods are provided: infrared debinding and laser debinding, which are advantageously much faster than traditional thermal debinding in a furnace.

After ADLP, the green body contains the doped ZrO₂ ceramic powders in the cured polymer resin, which is exposed to a debinding process to remove all the organic polymers (including the resin and dispersant). The debinding methods include but are not limited to: thermal debinding, infrared debinding, laser debinding, and plasma debinding. The dimensions of the debound “brown body” of dental crowns are from about 1×1×1 mm³ to about 50×50×50 mm³.

In the conventional thermal debinding process, the organic polymers are removed due to their decomposition at elevated temperatures. The green body is placed in a furnace and slowly heated up to about 800° C. The atmosphere includes but is not limited to: air, argon (Ar), nitrogen (N₂), and/or helium (He). The heating rate is from about 0.1 to 10° C./min, and the temperature is held at one or more temperatures (including but not limited to 750° C., 700° C., 650° C., 600° C., 550° C., 500° C., 450° C., 400° C., 350° C., 300° C., 250° C., 200° C., 150° C.) for 0.5 to 5 hours.

In the infrared debinding process, the organic polymers are removed by absorption and decomposition as a result of the direct interaction of infrared light with the polymers. Useful sources of infrared light may include but are not limited to: an infrared heater, an infrared emitter, an infrared lamp, an infrared light bulb, a halogen infrared lamp, an infrared light emitting diode, a Quartz infrared lamp, and a carbon infrared heater. The wavelength of infrared light is from about 700 nm to about 1 mm, the power is from about 1 to about 5,000 W, and the total debinding time is from about 1 minute to about 10 hours. The temperature is held at one or more temperatures between about 150° C. to about 750° C. (including but not limited to 750° C., 700° C., 650° C., 600° C., 550° C., 500° C., 450° C., 400° C., 350° C., 300° C., 250° C., 200° C., 150° C.) for 1 minute to 1 hour. The atmosphere includes but is not limited to: air, argon (Ar), nitrogen (N₂), and/or helium (He).

In the laser debinding process, the organic polymers are removed by thermal ablation and photo-ablation mechanisms as a result of the direct interaction of the laser beam with the polymers. Useful types of lasers for laser debinding may include but are not limited to: continuous-wave CO₂ lasers, pulsed lasers (such as Nd:YAG lasers, Er:YAG lasers, excimer lasers), fiber lasers, and diode lasers. The laser wavelength is from about 100 to about 3,000 nm, the laser power density is from about 1 to about 100 W/cm², and the total debinding time is from about 1 minute to about 1 hour. The atmosphere includes but is not limited to: air, argon (Ar), nitrogen (N₂), and/or helium (He).

### Laser Sintering and Electric Field Sintering

According to certain embodiments, any of multiple sintering methods may be used including two new sintering methods: laser sintering and electrical field sintering, which are much faster than the traditional pressureless sintering in a furnace. The dimensions of the sintered dental crowns are typically from about 1×1×1 mm³ to about 40×40×40 mm³.

After debinding, the porous brown body contains only the doped ZrO₂ ceramics, which needs to be sintered to become fully densified specimens (>99% relative density) that have the maximum mechanical performance (including hardness, compressive strength, flexural strength, and fracture toughness). The sintering methods include but are not limited to: pressureless sintering, laser sintering, electrical field sintering, spark plasma sintering, and microwave sintering.

In the conventional pressureless sintering process, densification of the brown body of doped ZrO₂ ceramics occurs by solid-state diffusion that transports matter from grain boundaries into the pores. The rate of matter transport is determined by atomic diffusion along the grain boundaries. Pressureless sintering of ZrO₂ ceramics requires high temperatures and a long time, and may need sintering additives due to the strong ionic and covalent bonds and low self-diffusion coefficients. The atmosphere includes but is not limited to: air, argon (Ar), nitrogen (N₂), and/or helium (He). The heating and cooling rate are from about 1 to 100° C./min, and the temperature is held at one or more temperatures (including but is not limited to 1200° C., 1250° C., 1300° C., 1350° C., 1400° C., 1450° C., 1500° C., 1550° C., 1600° C., 1650° C., 1700° C.) for 0.5 to 10 hours.

In the laser sintering process. a high-energy laser beam is used to accomplish fast sintering (in a few seconds to minutes) of the brown body of doped ZrO₂ ceramics at a high temperature (>1500° C.) and high heating/cooling rates (102-104° C./s). The high heating rate can suppress the grain growth while the high energy density of the laser beam can provide a high driving force for the sintering process through grain-boundary diffusion at high temperatures. Useful types of lasers for laser sintering include but are not limited to: continuous-wave CO₂ lasers, pulsed lasers (such as Nd:YAG lasers, Er:YAG lasers, excimer laser)s, Yb fiber lasers, and diode lasers. The laser wavelength is from about 100 to 3,000 nm, the laser power density is from about 1 to about 10,000 W/cm², and the sintering time is from about 1 second to about 30 minutes. The atmosphere includes but is not limited to: air, argon (Ar), nitrogen (N₂), and/or helium (He).

In the electrical field sintering process, an electrical field is applied to the brown body of doped ZrO₂ ceramics. Fast sintering (in a few seconds to minutes) occurs abruptly at a threshold temperature for a given applied voltage. The types of electric power include both direct current (DC) and alternating current (AC) powers. The atmosphere includes but is not limited to: air, argon (Ar), nitrogen (N₂), and/or helium (He). The electrical field is from about 10 to about 200 V/cm. The threshold temperature is from about 300 to about 1500° C. The sintering time is from about 1 second to about 30 minutes. The atmosphere includes but is not limited to: air, argon (Ar), nitrogen (N₂), and/or helium (He).

### Laser Polishing and Laser Shock Peening

According to certain embodiments, laser polishing and/or laser shock peening are applied to the ceramic dental crowns, which can make their surface more smooth and fatigue resistant.

After the sintering, surface engineering can be applied to improve the surface microstructures and mechanical performance of doped ZrO₂ ceramic dental crowns. The methods of surface engineering include but are not limited to: mechanical polishing, laser polishing, laser shock peening, shot peening, and water jet peening.

Ultrafast femtosecond (fs) lasers may be used to reduce the surface roughness and remove the open porosity in the doped ZrO₂ ceramic dental crowns. The extremely high peak power together with the ultra-short pulse duration can suppress the thermal effects to avoid thermal stress and cracking. Useful types of lasers for laser polishing may include but are not limited to diode-pumped fs lasers and fiber lasers. The laser wavelength is from about 100 to 3,000 nm. The laser power is from about 0 to about 100 W, e.g., from about 0.01 W to about 100 W. The pulse duration is from about 1 to about 500 fs. The laser spot diameter is from about 0 to about 100 μm, e.g., from about 0.01 μm to about 100 μm.

LSP can increase the fracture and fatigue resistance of the dental crowns by introducing significant compressive residual stress on the surface. useful types of lasers for LSP may include but are not limited to pulsed lasers (such as Nd:YAG lasers, Er:YAG lasers, excimer lasers) and fiber lasers. The laser wavelength is from about 400 to 2,000 nm. The laser pulse energy is from about 0 to 2 J, e.g., from about 0.01 J to about 2 J, and the pulse duration is 1 to 30 nanoseconds. The laser spot diameter is from 0 to 5 mm, e.g., from about 0.01 mm to about 5 mm. The laser power intensity is from 0 to about 30 GW/cm², e.g., from about 0.01 GW/cm² to about 30 GW/cm². The processing temperature is from about 25° C. to about 1300° C.

### Supporting Results

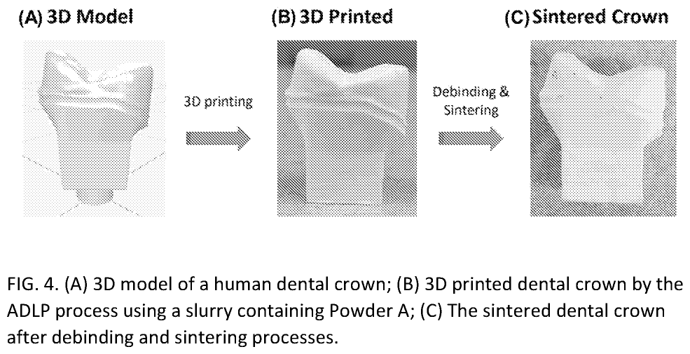

FIG. 4, panel (A) shows a 3D model of a human dental crown manufactured according to an embodiment. Initially, the STL file was imported into the ADLP system for 3D printing. A slurry containing 40 wt % Powder A, 8 wt % OA (dispersant), and Genesis base resin was prepared by magnetic stir mixing at room temperature. The ADLP system used a UV light source with a wavelength of 405 nm. The dental crowns (FIG. 4, panel (B)) were successfully 3D printed at room temperature. It can be seen that the 3D printed dental crowns exhibit most of the morphology features of the model, a smooth surface condition, and white and opaque color. After debinding at 200° C. to 550° C. and sintering at 1500° C., ZrO₂-based ceramic dental crowns were successfully formed (FIG. 4, panel (C)).

The debinding experiments using an infrared lamp suggested that the organic polymers in the green body can be completely removed by the infrared light irradiation in about half an hour in contrast to over 20 hours by thermal debinding in a furnace.

The physical properties of the sintered 3D printed objects using Powder A and B were measured and compared to the international standard (Table 1). The sintered 3D objects have over 97% of the theoretical density, and have a very high hardness to ensure an excellent wear resistance. The international standard ISO 13356:2015 specifies the requirements for Y-TZP as a material for surgical implants. The density, hardness, and strength of the sintered ZrO₂-based ceramic dental crowns are close to or exceed the requirements in the ISO 13356:2015 standard.

TABLE 1
Physical properties of the sintered 3D printed
objects using Powder A and Powder B, respectively,
compared to the ISO 13356: 2015 standard.

| | Sintered 3D printed objects | | |
|---|---|---|---|
| | from: | | ISO |
| | Powder A | Powder B | 13356: 2015 |
| Density (g/cm³) | 6.02 | 5.92 ± 0.12 | >6.0 |
| Vickers hardness at 1 kg | 14.2 ± 0.6 | 14.4 ± 0.1 | >11.8 |
| (GPa) | | | |
| Bending strength (MPa) | 766 ± 170 | — | >800 |
| Surface roughness (Ra - | 3.48 ± 2.54 | 3.03 ± 1.92 | — |
| μm) | | | |
| Light transmittance (%) | 26.0 ± 2.3 | 33.3 ± 2.4 | — |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the disclosed subject matter (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or example language (e.g., “such as”) provided herein, is intended merely to better illuminate the disclosed subject matter and does not pose a limitation on the scope of the invention unless otherwise claimed. No language in the specification should be construed as indicating any non-claimed element as essential to the practice of the invention.

Certain embodiments are described herein. Variations of those embodiments may become apparent to those of ordinary skill in the art upon reading the foregoing description. The inventors expect skilled artisans to employ such variations as appropriate, and the inventors intend for the embodiments to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A method of manufacturing a ZrO₂-based ceramic dental crown, the method comprising:
mixing a doped ZrO₂ powder with a solvent and a dispersant to produce a slurry;
drying the slurry to remove the solvent and produce a dried ceramic powder;
mixing the dried ceramic powder with a photo-curable resin to produce a printing mixture; and
3D printing a dental crown green body structure using the printing mixture, wherein the 3D printing includes an advanced digital light processing (ADLP) process, and
wherein the doped ZrO₂ powder is selected from the group consisting of Powder A, Powder B, Powder C and Powder D,
wherein Powder A contains 2 to 4 mol. % yttrium (Y)-doped zirconia,
wherein Powder B contains 4 to 8 mol. % yttrium (Y)-doped zirconia,
wherein Powder C contains zirconia doped with 2 to 4 mol. % yttrium (Y) and 0.1 to 1.0 mol. % iron (Fe), and
wherein Powder D contains zirconia doped with one or more of magnesium (Mg), calcium (Ca), cerium (Ce), aluminum (Al), titanium (Ti), germanium (Ge), scandium (Sc), or tin (Sn).

2. The method of claim 1, wherein the photo-curable resin is selected from the group consisting of Genesis flexible development base resin (Tethon 3D), 1,6-hexanediol diacrylate (HDDA), polyethylene glycol diacrylate (PEGDA), and diphenyl (2,4,6-trimethylbenzoyl) phosphine oxide (TPO).

3. The method of claim 1, wherein the dispersant is selected from the group consisting of 1-10 wt. % oleic acid (OA), DISPERBYK-103, DISPERBYK-111, DISPERBYK-180, DISPERBYK-2008, DISPERBYK-2013, DISPERBYK-2155, TEGO Dispers 685, and polyvinyl pyrrolidone K15 (PVP-K15).

4. The method of claim 1, wherein the 3D printing includes a gradient printing process.

5. The method of claim 1, wherein the method further includes:
debinding the dental crown green body structure to remove organic polymers, including the resin and the dispersant, and to produce a debound dental crown structure using one of a thermal debinding process, an infrared debinding process, and a laser debinding process.

6. The method of claim 5, wherein the method further includes:
sintering the debound dental crown structure to produce a densified dental crown structure using one of a pressureless sintering process, a laser sintering process, and an electric field sintering process.

7. The method of claim 6, wherein the method further includes:
surface engineering the densified dental crown structure to produce a finished dental crown structure using one of a mechanical polishing process, a laser polishing process, a laser shock peening process, a shot peening process, and a water jet process.

8. The method of claim 1, wherein the ADLP process includes printing the dental crown green body structure using two or more different printing mixtures, each different printing mixture including one of Powder A, Powder B, Powder C or Powder D, using a gradient printing technique to form a color gradient in the dental crown green body structure.

9. An ADLP system for manufacturing a ZrO₂-based ceramic object, the ADLP system comprising:
a resin tank structure including at least two separate resin vats, each of the at least two separate resin vats holding a different printing mixture comprising a photo-curable resin and a different doped ZrO₂ powder;
a build platform configured to hold a green body object during a printing operation;
movement components configured to controllably move the build platform relative to the resin tank structure in vertical and horizontal directions; and
a UV light source configured to project a UV light pattern through a bottom portion of the resin vat structure,
wherein during the printing operation, a first layer of the green body object is cured in a first one of the at least two separate resin vats, and a second layer of the green body object is cured in a second one of the at least two separate resin vats.

10. The ADLP system of claim 9, wherein the movement components include an elevator structure configured to move the build platform in the vertical direction, and a resin tank guide structure configured to move the resin tank structure in a horizontal direction.

11. The ADLP system of claim 9, further including a controller configured to control the movement components and the UV light source.

12. The ADLP system of claim 9, wherein the resin tank structure includes at least three separate resin holding vats.

13. The ADLP system of claim 9, wherein each different doped ZrO₂ powder includes a doped ZrO₂ powder selected from the group consisting of Powder A, Powder B, Powder C and Powder D,
wherein Powder A contains 2 to 4 mol. % yttrium (Y)-doped zirconia,
wherein Powder B contains 4 to 8 mol. % yttrium (Y)-doped zirconia,
wherein Powder C contains zirconia doped with 2 to 4 mol. % yttrium (Y) and 0.1 to 1.0 mol. % iron (Fe), and
wherein Powder D contains zirconia doped with one or more of magnesium (Mg), calcium (Ca), cerium (Ce), aluminum (Al), titanium (Ti), germanium (Ge), scandium (Sc), and or tin (Sn).

14. The ADLP system of claim 9, wherein the photo-curable resin is selected from the group consisting of Genesis flexible development base resin (Tethon 3D), 1,6-hexanediol diacrylate (HDDA), polyethylene glycol diacrylate (PEGDA), and diphenyl (2,4,6-trimethylbenzoyl) phosphine oxide (TPO).

15. The ADLP system of claim 9, wherein the green body object is a dental crown green body structure.
